(19) United States
(12) Patent Application Publication
(10) Pub. No.: US 2025/0078255 A1
(43) Pub. Date: Mar. 6, 2025

(54) ENDOSCOPIC EXAMINATION SUPPORT APPARATUS, ENDOSCOPIC EXAMINATION SUPPORT METHOD, AND RECORDING MEDIUM

(71) Applicant: NEC CORPORATION, Minato-ku, Tokyo (JP)

(72) Inventor: Atsushi MARUGAME, Tokyo (JP)

(73) Assignee: NEC CORPORATION, Minato-ku, Tokyo (JP)

(21) Appl. No.: 18/288,715

(22) PCT Filed: Nov. 25, 2022
(86) PCT No.: PCT/JP2022/043467

§ 371 Date: Oct. 27, 2023

(51) Int. Cl. G06T 7/00; G06T 7/73; G16H 50/30

(52) U.S. Cl. CPC G06T 7/0012; G06T 7/75; G16H 50/30; G06T 2207/10068; G06T 2207/20081; G06T 2207/30028; G06T 2207/30096

(57) ABSTRACT

In the endoscopic examination support apparatus, the lesion candidate detection means detects a lesion candidate from an endoscopic image acquired by imaging an interior of a luminal organ. The flag setting means sets a flag according to a position in the interior of the luminal organ at which the endoscopic image including the lesion candidate is acquired. The risk determination means determines a risk of the lesion candidate based on at least a size of the lesion candidate and the flag. The display image generation means generates a display image including information relating to a position of the lesion candidate in the endoscopic image, information relating to a position in the interior of the luminal organ corresponding to the flag, and information relating to the risk of the lesion candidate.

### TECHNICAL FIELD

The present disclosure relates to a technique for presenting information to support endoscopic examination.

### BACKGROUND ART

As information for supporting the endoscopic examination performed on a living body, there is conventionally known a technique for presenting information of the position of the attention region such as a lesion within the living body.

Specifically, for example, Patent Document 1 discloses a technique for storing the position of the attention area, such as a lesion candidate detected by a capsule-type endoscope passing through the body of the patient, and displaying the position of the attention area on an intrabody map during examination of the body of the patient by the scope-type endoscope.

### PRECEDING TECHNICAL REFERENCES PATENT DOCUMENT

Patent Document 1: Japanese Patent Application Laid-Open under No. 2011-156203

### SUMMARY

### Problem to be Solved

However, according to the technique disclosed in Patent Document 1, there is such a problem that, in order to display the position of the lesion candidate at the time of the examination by the scope-type endoscope, the examination by the capsule-type endoscope must be performed in advance. That is, according to the technique disclosed in Patent Document 1, there is such a problem that the procedure for displaying information relating to the lesion candidates at the time of endoscopic examination becomes complicated.

It is an object of the present disclosure to provide an endoscopic examination support apparatus capable of easily displaying information related to lesion candidates at the time of the endoscopic examination.

### Means for Solving the Problem

According to an example aspect of the present invention, there is provided an endoscopic examination support apparatus comprising:
a lesion candidate detection means configured to detect a lesion candidate from an endoscopic image acquired by imaging an interior of a luminal organ;
a flag setting means configured to set a flag according to a position in the interior of the luminal organ at which the endoscopic image including the lesion candidate is acquired;
a risk determination means configured to determine a risk of the lesion candidate based on at least a size of the lesion candidate and the flag; and
a display image generation means configured to generate a display image including information relating to a position of the lesion candidate in the endoscopic image, information relating to a position in the interior of the luminal organ corresponding to the flag, and information relating to the risk of the lesion candidate.

According to another example aspect of the present invention, there is provided an endoscopic examination support method comprising:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate.

According to still another example aspect of the present invention, there is provided a recording medium storing a program, the program causing a computer to execute:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate.

### Effect

According to the present disclosure, it is possible to easily display information relating to a lesion candidate at the time of endoscopic examination.

### BRIEF DESCRIPTION OF THE DRAWINGS

FIG. 1 is a diagram showing a schematic configuration of an endoscopic examination system according to a first example embodiment.

FIG. 2 is a block diagram showing a hardware configuration of an endoscopic examination support apparatus according to the first example embodiment.

FIG. 3 is a block diagram showing a functional configuration of the endoscopic examination support apparatus according to the first example embodiment.

FIG. 4 is a diagram illustrating an example of table data used for processing the endoscopic examination support apparatus according to the first example embodiment.

FIG. 5 is a diagram illustrating another example of table data used for processing the endoscopic examination support apparatus according to the first example embodiment.

FIG. 6 is a diagram illustrating an example of a display image generated by the processing of the endoscopic examination support apparatus according to the first example embodiment.

FIG. 7 is a flowchart illustrating an example of processing performed by the endoscopic examination support apparatus according to the first example embodiment.

FIG. 8 is a block diagram showing a functional configuration of an endoscopic examination support apparatus according to a second example embodiment.

FIG. 9 is a flowchart for explaining processing performed by the endoscopic examination support apparatus according to the second example embodiment.

### EXAMPLE EMBODIMENTS

Preferred example embodiments of the present disclosure will be described with reference to the accompanying drawings.

### First Example Embodiment

### [System Configuration]

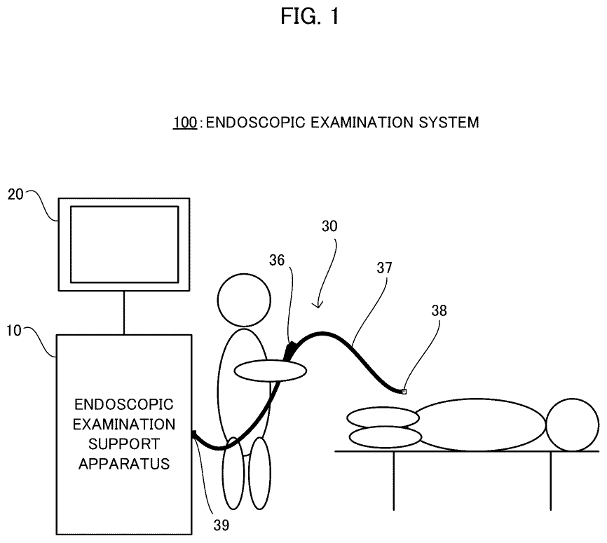

FIG. 1 is a diagram showing a schematic configuration of an endoscopic examination system according to a first example embodiment. As shown in FIG. 1, the endoscopic examination system 100 includes an endoscopic examination support apparatus 10, a display device 20, and an endoscope 30 connected to the endoscopic examination support apparatus 10.

The endoscopic examination support apparatus 10 acquires a video including time-series images acquired by imaging a subject during an endoscopic examination (hereinafter, also referred to as “endoscopic video Ic”) from the endoscope 30, and displays a display image for monitoring by an operator such as a doctor performing an endoscopic examination on the display device 20. Specifically, the endoscopic examination support apparatus 10 acquires a video of the interior of the colon obtained during the endoscopic examination from the endoscope 30 as the endoscopic video Ic. The endoscopic examination support apparatus 10 detects a lesion candidate area that is an area including the lesion candidate in the image extracted from the endoscopic video Ic (hereinafter, also referred to as “endoscopic image”). The endoscopic examination support apparatus 10 performs classification according to the size of the lesion candidate included in the lesion candidate area. Also, the endoscopic examination support apparatus 10 performs classification according to the type of the lesion candidate included in the lesion candidate area. The endoscopic examination support apparatus 10 performs a determination of the current position of the endoscope camera in the colon based on the endoscopic image. In addition, the endoscopic examination support apparatus 10 acquires risk information that is information related to the determination result obtained by determining the risk of the lesion candidate, on the basis of the position in the colon at which the endoscopic image including the lesion candidate is acquired, the classification result according to the size of the lesion candidate, and the classification result according to the type of the lesion candidate. Further, the endoscopic examination support apparatus 10 generates a display image including the information relating to the position of the lesion candidate in the endoscopic image, the information relating to the position in the colon at which the lesion candidate is detected, and the information relating to the risk of the lesion candidate, and outputs the generated display image to the display device 20.

The display device 20 may include a liquid crystal display, for example. The display device 20 displays an image outputted from the endoscopic examination support apparatus 10.

The endoscope 30 mainly includes an operation unit 36 used by an operator to input instructions such as air supply, water supply, angle adjustment, and an image-capturing instruction, a shaft 37 having flexibility and inserted into an organ of a subject to be examined, a tip portion 38 with a built-in imaging unit such as an ultra-compact imaging element, and a connection unit 39 for connection with the endoscopic examination support apparatus 10. The endoscope 30 can acquire the endoscopic image by imaging the interior of the colon which is a luminal organ.

### [Hardware Configuration]

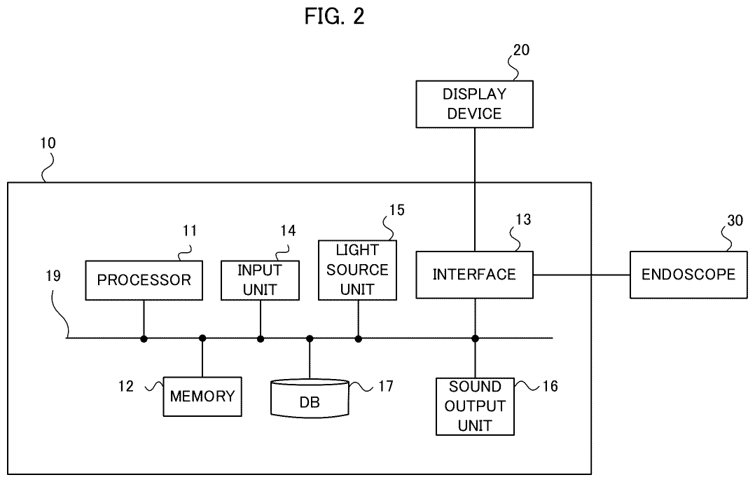

FIG. 2 shows a hardware configuration of the endoscopic examination support apparatus. The endoscopic examination support apparatus 10 mainly includes a processor 11, a memory 12, an interface 13, an input unit 14, a light source unit 15, a sound output unit 16, and a data base (hereinafter referred to as “DB”) 17. Each of these elements is connected via a data bus 19.

The processor 11 executes a predetermined processing by executing a program stored in the memory 12. The processor 11 is a processor such as a CPU (Central Processing Unit), a GPU (Graphics Processing Unit), and a TPU (Tensor Processing Unit). The processor 11 may be configured by a plurality of processors. The processor 11 is an example of a computer. Also, the processor 11 executes processing for generating the display image.

The memory 12 is configured by various volatile memories used as a working memory and non-volatile memories for storing information needed for processing the endoscopic examination support apparatus 10, such as a RAM (Random Access Memory) and a ROM (Read Only Memory). Incidentally, the memory 12 may include an external storage device such as a hard disk connected to or incorporated in the endoscopic examination support apparatus 10, and may include a storage medium such as a removable flash memory or a disk medium. The memory 12 stores a program for the endoscopic examination support apparatus 10 to execute each process in the present example embodiment.

Also, the memory 12 temporarily stores a series of endoscopic videos Ic captured by the endoscope 3 in the endoscopic examination, based on the control of the processor 11.

The interface 13 performs an interface operation between the endoscopic examination support apparatus 10 and the external devices. For example, the interface 13 supplies the display image generated by the processor 11 to the display device 20. Also, the interface 13 supplies the illumination light generated by the light source unit 15 to the endoscope 30. Also, the interface 13 supplies an electrical signal indicating the endoscopic video Ic supplied from the endoscope 30 to the processor 11. The interface 13 may be a communication interface such as a network adapter for wired or wireless communication with an external device, or may be a hardware interface compliant with a USB (Universal Serial Bus), SATA (Serial Advanced Technology Attachment), etc.

The input unit 14 generates an input signal based on the operation of the operator. The input unit 14 is, for example, a button, a touch panel, a remote controller, a voice input device, or the like. The light source unit 15 generates the light to be delivered to the tip portion 38 of the endoscope 30. The light source unit 15 may also incorporate a pump or the like for delivering water or air to be supplied to the endoscope 30. The sound output unit 16 outputs the sound based on the control of the processor 11.

The DB 17 stores the endoscopic images acquired by past endoscopic examinations of the subject. The DB 17 may include an external storage device, such as a hard disk connected to or incorporated in the endoscopic examination support device 10, and may include a storage medium, such as a removable flash memory. Instead of providing the DB 17 in the endoscopic examination system 100, the DB 17 may be provided in an external server or the like to acquire associated information from the server through communication.

Incidentally, the endoscopic examination support apparatus 10 may be provided with a sensor capable of measuring the rotation and translation of the endoscope camera, such as a magnetic sensor.

### [Functional Configuration]

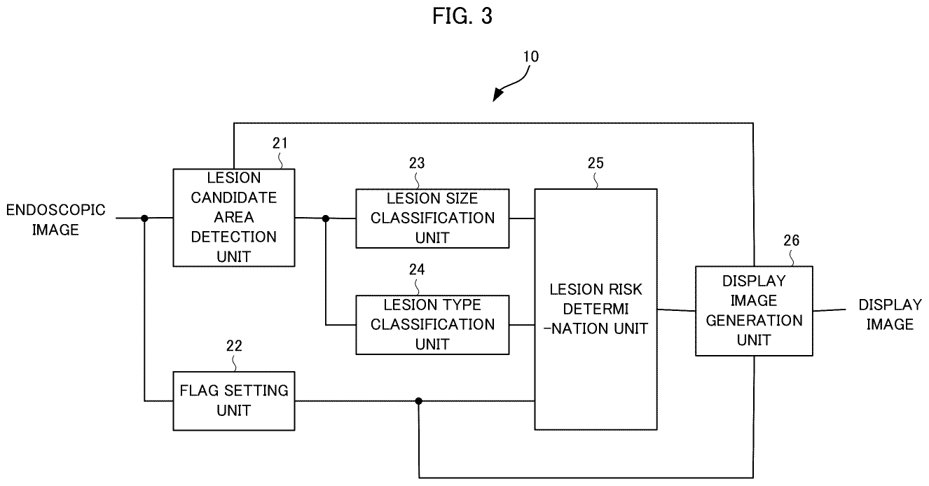

FIG. 3 is a block diagram illustrating a functional configuration of the endoscopic examination support apparatus according to the first example embodiment. As shown in FIG. 3, the endoscopic examination support apparatus 10 includes a lesion candidate area detection unit 21, a flag setting unit 22, a lesion size classification unit 23, a lesion type classification unit 24, a lesion risk determination unit 25, and a display image generation unit 26.

The lesion candidate area detection unit 21 has a function as a lesion candidate detection means. The lesion candidate area detection unit 21 detects the lesion candidate areas in the endoscopic image. That is, the lesion candidate area detection unit 21 can detect the lesion candidates from the endoscopic images acquired by imaging the inside of the colon. Incidentally, the detection of the lesion candidate area may be performed by image processing of the endoscopic image, or it may be performed by a user who visually monitors the endoscopic image. Then, the lesion candidate area detection unit 21 adds the detection result of the lesion candidate area to the endoscopic image, and outputs the endoscopic image to the lesion size classification unit 23, the lesion type classification unit 24 and the display image generation unit 26. The detection result of the lesion candidate area may include at least information indicating the position of the lesion candidate area in the endoscopic image.

The flag setting unit 22 has a function as a flag setting means. The flag setting unit 22 performs determination of the current position of the endoscope camera in the colon on the basis of the same endoscopic image as that inputted to the lesion candidate area detection unit 21. Then, the flag setting unit 22 sets a flag corresponding to the determination result of the current position of the endoscope camera in the colon, and outputs the flag information including the flag to the lesion risk determination unit 25 and the display image generation unit 26.

Here, a specific example of the function of the flag setting unit 22 will be described.

The flag setting unit 22 includes, for example, a discriminator DK that is trained to output data used to identify the position in the colon corresponding to the image including a subject in the colon in response to the input of the image, and a timer TA capable of measuring an elapsed time from the starting time of endoscopic examination. In this case, the flag setting unit 22 can detect the current position of the endoscope camera based on the output data obtained by inputting the endoscopic image to the discriminator DK. The flag setting unit 22 may use, as the start time of the endoscopic examination, the time when it is first detected in one endoscopic examination that the current position of the endoscope camera is at or near the anus, for example.

Based on the current position of the endoscope camera and the elapsed time measured by the timer TA, the flag setting unit 22 determines whether or not it corresponds to a pull-out starting state in which the endoscope camera starts moving in the anal direction after reaching the cecum. Specifically, when the elapsed time measured by the timer TA is shorter than a predetermined time T1, the flag setting unit 22 acquires the determination result indicating that it does not correspond to the pull-out starting state, sets “0” to the flag corresponding to the determination result, and outputs the flag information including the flag to the lesion risk determination unit 25 and the display image generation unit 26. Further, when the elapsed time measured by the timer TA is equal to or longer than the predetermined time T1 and the endoscope camera has not reached the cecum, the flag setting unit 22 acquires the determination result indicating that it does not correspond to the pull-out starting state, sets “0” to the flag indicating the determination result, and outputs the flag information including the flag to the lesion risk determination unit 25 and the display image generation unit 26. When the elapsed time measured by the timer TA is equal to or longer than the predetermined time T1 and it is detected for the first time that the endoscope camera has reached the cecum in one endoscopic examination, the flag setting unit 22 acquires the determination result indicating that it corresponds to the pull-out starting state.

In the period after the timing of obtaining the determination result indicating that it corresponds to the pull-out starting state (hereinafter, also referred to as “pull-out period”), the flag setting unit 22 performs a determination of whether or not the endoscope camera has passed through the splenic curvature, based on the detection result of the current position of the endoscope camera. In such a determination, it is desirable that a predetermined position in the splenic curvature is used as the determination reference position, for example.

When the flag setting unit 22 acquires the determination result indicating that the endoscope camera has not passed through the splenic curvature during the pull-out period, the flag setting unit 22 sets “1” as a flag corresponding to the determination result, and outputs the flag information including the flag to the lesion risk determination unit 25 and the display image generation unit 26. The flag “1” indicates that the endoscopic image subjected to the detection of the lesion candidate has been acquired in one of the cecum, the ascending colon, and the transverse colon, which are the parts belonging to the right side of the colon. When the flag setting unit 22 acquires the determination result indicating that the endoscope camera has passed through the splenic curvature during the pull-out period, the flag setting unit 22 sets “2” as a flag corresponding to the determination result, and outputs the flag information including the flag to the lesion risk determination unit 25 and the display image generation unit 26. The flag “2” indicates that an endoscopic image subjected to the detection of the lesion candidate has been acquired in one of the descending colon, sigmoid colon, or rectum, which are the parts belonging to the left side of the colon.

According to the above-described specific example, the flag setting unit 22 can set the flag corresponding to the position in the colon at which the endoscopic image including the lesion candidate is acquired. Also, the flag setting unit 22 can set the flag “1” in a period from the time when the pull-out of the endoscope camera inserted into the colon to obtain the endoscopic image is started, to the time when the endoscope camera passes through the predetermined site in the colon, and can set the flag “2” in a period from the time when the endoscope camera passes through the predetermined site, to the time when the pull-out of the endoscope camera is completed. In addition, the flag setting unit 22 can set the flag “1” in a period from the time when the endoscope camera reaches the cecum to the time when the endoscope camera passes through the splenic curvature, and can set the flag “2” in a period from the time when the endoscope camera passes through the splenic curvature to the time when the endoscope camera passes through the anus.

The lesion size classification unit 23 classifies the size of the lesion candidate included in the lesion candidate area of the endoscopic image, based on the endoscopic image outputted from the lesion candidate area detection unit 21. Then, the lesion size classification unit 23 outputs the size information which is information relating to the classification result of the size of the lesion candidate to the lesion risk determination unit 25.

Here, a specific example of the function of the lesion size classification unit 23 will be described.

The lesion size classification unit 23 includes a discriminator DL which is trained to output an output data capable of specifying the size of the lesion in response to the input of an image acquired by imaging a lesion within the colon, for example. In that case, the lesion size classification unit 23 classifies the size of the lesion candidate included in the lesion candidate area into one of a plurality of sizes, based on the output data obtained by inputting a rectangular image cut out from the endoscopic image in accordance with the detection result of the lesion candidate area to the discriminator DL. In the above-described case, it is desirable that the lesion size classification unit 23 inputs a rectangular image deformed to have a size and an aspect ratio suitable for classification of the sizes of the lesion candidates to the discriminator DL. Further, in the the above case, the lesion size classification unit 23 can output information indicating that the lesion candidate included in the lesion candidate area is classified into one of the sizes of “large”, “medium”, and “small” to the lesion risk determination unit 25 as the size information, for example. Specifically, the lesion size classification unit 23 can output, as the size information, information indicating that the lesion candidates equal to or larger than 10 mm are classified into the size “large”, information indicating that the lesion candidates equal to or larger than 5 mm and smaller than 10 mm are classified into the size “medium”, and information indicating that the lesion candidates smaller than 5 mm are classified into the size “small”.

According to the above-described specific example, the lesion size classification unit 23 may classify the lesion candidates having the same or similar size to each other into the same category. Also, according to the above-described specific example, when the risk determination of the lesion candidate described later is performed, the lesion candidates classified into one of the categories of the size “large”, “medium” and “small” are treated as having the same size to each other.

The lesion type classification unit 24 classifies the type of the lesion candidate included in the lesion candidate area of the endoscopic image based on the endoscopic image outputted from the lesion candidate area detection unit 21. Then, the lesion size classification unit 23 outputs the type information, which is information relating to the classification result of the type of the lesion candidate, to the lesion risk determination unit 25.

Here, a specific example of the function of the lesion type classification unit 24 will be described.

The lesion type classification unit 24 includes a discriminator DM which is trained to output data capable of specifying the type of the lesion in response to the input of the image acquired by imaging the lesion in the colon, for example. In such a case, the lesion type classification unit 24 can classify the type of the lesion candidate included in the lesion candidate area into one of the plurality of types based on the output data obtained by inputting the rectangular image cut out from the endoscopic image according to the detection result of the lesion candidate area to the discriminator DM. In the above-described case, it is desirable that the lesion type classification unit 24 inputs the rectangular image that is deformed to have a size and an aspect ratio suitable for classification of the type of the lesion candidate into the discriminator DM. Further, in the case as described above, the lesion type classification unit 24 can output information indicating that the lesion candidates included in the lesion candidate area are classified into any type of “tumor”, “serrated lesion” and “others” to the lesion risk determination unit 25 as the type information, for example. Further, in the case as described above, the lesion type classification unit 24 can output information indicating that the lesion candidates included in the lesion candidate area are classified into any type of “adenoma”, “adenocarcinoma”, “serrated lesion” and “others” to the lesion risk determination unit 25 as the type information, for example.

The lesion risk determination unit 25 has a function as a risk determination means. The lesion risk determination unit 25 determines the risk of the lesion candidate included in the endoscopic image based on the flag information outputted from the flag setting unit 22, the size information outputted from the lesion size classification unit 23, and the type information outputted from the lesion type classification unit 24. Then, the lesion risk determination unit 25 outputs the risk information, which is information relating to the determination result of the risk of the lesion candidates, to the display image generation unit 26.

Here, a specific example of the function of the lesion risk determination unit 25 will be described.

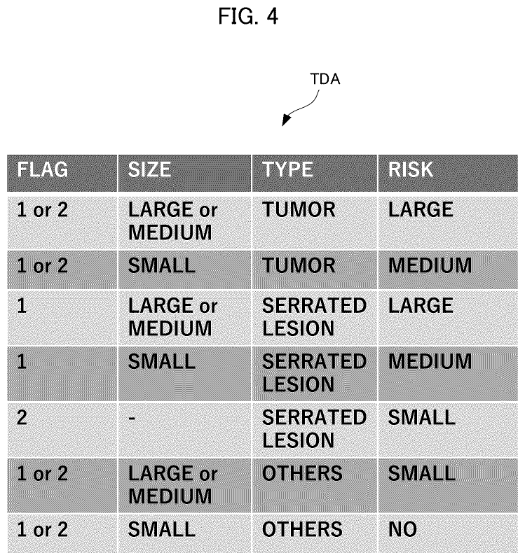

FIG. 4 is a diagram showing an example of table data used for the processing of the endoscopic examination support device according to the first example embodiment. The lesion risk determination unit 25 determines the risk of the lesion candidate based on the table data TDA shown in FIG. 4, the flag included in the flag information, the size of the lesion candidate included in the size information, and the type of the lesion candidate included in the type information. Specifically, for example, when the flag included in the flag information is “1”, the size of the lesion candidate included in the size information is “large” or “medium”, and the type of the lesion candidate included in the type information is “serrated lesion”, the lesion risk determination unit 25 determines that the risk of the lesion candidate is “large”. For example, when the flag included in the flag information is “1”, the size of the lesion candidate included in the size information is “small”, and the type of the lesion candidate included in the type information is “serrated lesion”, the lesion risk determination unit 25 determines that the risk of the lesion candidate is “medium”. For example, when the flag included in the flag information is “2” and the type of the lesion candidate included in the type information is “serrated lesion”, the lesion risk determination unit 25 determines that the risk of the lesion candidate is “small”. Further, for example, when the flag included in the flag information is “1” or “2”, the size of the lesion candidate included in the size information is “small”, and the type of the lesion candidate included in the type information is “others”, the lesion risk determination unit 25 determines that the risk of the lesion candidate is “no”. That is, by performing the processing using the table data TDA, the lesion risk determination unit 25 can determine which one of “large”, “medium”, “small” and “no” the risk of the lesion candidates included in the endoscopic image corresponds to.

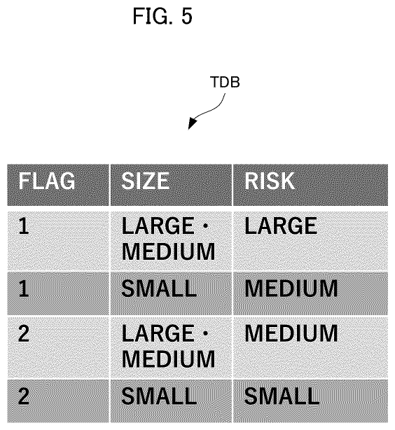

Instead of the table data TDA, the lesion risk determination unit 25 may determine the risk of the lesion candidates using the table data TDB shown in FIG. 5. In such a case, the lesion risk determination unit 25 can determine the risk of the lesion candidate without using the type information. Specifically, for example, when the flag included in the flag information is “1” and the size of the lesion candidate included in the size information is “large” or “medium”, the lesion risk determination unit 25 determines that the risk of the lesion candidate is “large”. For example, when the flag included in the flag information is “1” and the size of the lesion candidate included in the size information is “small”, the lesion risk determination unit 25 determines that the risk of the lesion candidate is “medium”. For example, when the flag included in the flag information is “2” and the size of the lesion candidate included in the size information is “small”, the lesion risk determination unit 25 determines that the risk of the lesion candidate is “small”. That is, by performing the processing using the table data TDB, the lesion risk determination unit 25 can determine which one of “large”, “medium” and “small” the risk of the lesion candidates included in the endoscopic image corresponds to.

According to the above-described specific example, the lesion risk determination unit 25 can determine the risk of the lesion candidate based on at least the size of the lesion candidate indicated by the size information and the flag included in the flag information. In addition, when the size of the first lesion candidate included in the endoscopic image corresponding to the flag “1” and the size of the second lesion candidate included in the endoscopic image corresponding to the flag “2” are equivalent, the lesion risk determination unit 25 can determine that the risk of the first lesion candidate is not smaller than than the risk of the second lesion candidate. In addition, when the size of the above-described first lesion candidate is equal to the size of the above-described second lesion candidate, and the type of the above-described first lesion candidate is the same as the type of the above-described second lesion candidate, the lesion risk determination unit 25 can determine that the risk of the above-described first lesion candidate is not smaller than the risk of the above-described second lesion candidate.

The display image generation unit 26 has a function as a display image generation means. The display image generation unit 26 generates a display image based on the endoscopic image outputted from the lesion candidate area detection unit 21, the flag information outputted from the flag setting unit 22, and the risk information outputted from the lesion risk determination unit 25, and outputs the generated display image to the display device 20.

Here, a specific example of the function of the display image generation unit 26 will be described.

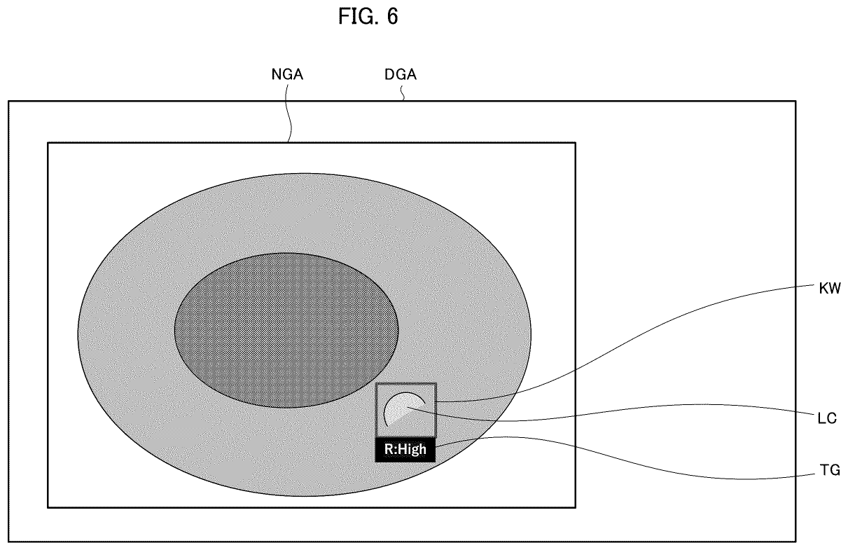

The display image generation unit 26 generates a display image DGA including an endoscopic image NGA as illustrated in FIG. 6 on the basis of the endoscopic image outputted from the lesion candidate area detection unit 21, the detection result of the lesion candidate area added to the endoscopic image, the flag information outputted from the flag setting unit 22, and the risk information outputted from the lesion risk determination unit 25. FIG. 6 is a diagram illustrating an example of a display image generated by the processing of the endoscopic examination support apparatus according to the first example embodiment.

The endoscopic image NGA is generated, for example, by adding the information visually indicating the position of the lesion candidate LC in the endoscopic image, the information visually indicating the position in the colon (right side or left side of the colon) at which the lesion candidate LC is detected, and the information visually indicating the risk of the lesion candidate LC, to the same endoscopic image as that inputted to the lesion candidate area detection unit 21. In addition, the endoscopic image NGA includes a detection frame KW surrounding the lesion candidate LC, and a tag TG provided at the lower portion of the detection frame KW.

For example, the detection frame KW is generated as a rectangular frame having the same size as the size of the lesion candidate area. Incidentally, the detection frame KW may be generated as a frame having a shape other than a rectangle (e.g., circle), as long as it surrounds the lesion candidate LC. The detection frame KW may be generated as a frame having a size different from the size of the lesion candidate area, as long as it surrounds the lesion candidate LC. The detection frame KW may be generated as a frame having a different line type according to the flag included in the flag information. Specifically, the detection frame KW may be generated as a frame of the solid line when the flag is “1” and may be generated as a frame of the dashed line when the flag is “2”, for example. The detection frame KW may be generated as a frame having a different color according to the risk included in the risk information. Specifically, the detection frame KW may be generated as a red frame when the risk is “large”, generated as a yellow frame when the risk is “medium”, generated as a green frame when the risk is “small”, and generated as a cyan frame when the risk is “no”, for example.

The tag TG includes at least one of one or more letters corresponding to the flag information and one or more letters corresponding to the risk information. The letter “R” in the tag TG of FIG. 6 corresponds to the flag “1” and indicates that the lesion candidate LC is detected on the right side of the colon. In addition, the letters “High” of the tag TG in FIG. 6 correspond to the risk “large” and indicate that the risk of the lesion candidates LC is high. According to this example embodiment, for example, in a case where the detection frame KW has a different line type according to the flag included in the flag information and the detection frame KW has a different color according to the risk included in the risk information, the tag TG may be omitted. Further, according to this example embodiment, for example, in a case where the detection frame KW has a different line type according to the flag included in the flag information, the tag TG without the letters corresponding to the flag information may be generated. Further, according to the present example embodiment, for example, in a case where the detection frame KW has a different color according to the risk included in the risk information, the tag TG without the letters corresponding to the risk information may be generated.

According to the above-described specific example, the display image generation unit 26 can generate a display image including the information relating to the position of the lesion candidate in the endoscopic image, the information relating to the position in the colon corresponding to the flag included in the flag information, and the information relating to the risk of the lesion candidate included in the risk information. Further, as a display image, the display image generation unit 26 can generate an image including the detection frame which surrounds the lesion candidate in the endoscopic image and is displayed in a different manner in accordance with the flag included in the flag information and the risk of the lesion candidate included in the risk information. Further, as the detection frame, the display image generation unit 26 can generate a frame having a different line type in accordance with the flag included in the flag information. Further, as the detection frame, the display image generation unit 26 can generate a frame having different colors according to the risk of the lesion candidate included in the risk information. Further, as the display image, the display image generation unit 26 can generate an image which includes a detection frame surrounding the lesion candidate present in the endoscopic image, the flag included in the flag information and the risk of the lesion candidate included in the risk information.

### [Processing Flow]

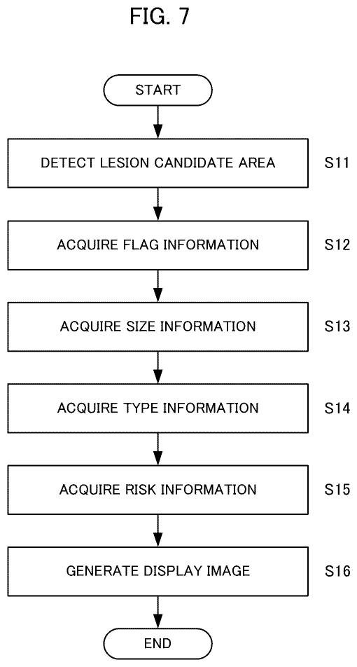

Subsequently, a flow of processing performed in the endoscopic examination support apparatus according to the first example embodiment will be described. FIG. 7 is a flowchart illustrating an example of processing performed in the endoscopic examination support apparatus according to the first example embodiment.

First, the endoscopic examination support device 10 detects the lesion candidate area in the endoscopic image (step S11).

Next, the endoscopic examination support apparatus 10 sets the flag by performing a determination relating to the present position of the endoscope camera in the colon based on the endoscopic image, and acquires the flag information including the set flag (step S12).

Subsequently, the endoscopic examination support apparatus 10 classifies the size of the lesion candidate included in the lesion candidate area detected in step S11 and acquires the size information relating to the classification result of the size of the lesion candidate (step S13).

Subsequently, the endoscopic examination support apparatus 10 classifies the type of the lesion candidate included in the lesion candidate area detected in step S11 and acquires the type information relating to the classification result of the type of the lesion candidate (step S14).

Subsequently, the endoscopic examination support apparatus 10 acquires the risk information relating to the determination result of the risk of the lesion candidate included in the lesion candidate area detected in step S11 by performing the determination based on the flag information obtained in step S12, the size information obtained in step S13, and the type information obtained in step S14 (step S15). In step S15, it is desirable that the determination is performed using the table data TDA or TDB described above.

Subsequently, the endoscopic examination support apparatus 10 generates a display image on the basis of the detection result of the lesion candidate area detected in step S11, the flag information obtained in step S12, and the risk information obtained in step S15 (step S16). According to the process of step S16, for example, it is possible to generate an image in which the information visually indicating the position of the lesion candidate in the endoscopic image, the information visually indicating the position (right side or left side of the colon) at which the lesion candidate is detected, and the information visually indicating the risk of the lesion candidate are added to the endoscopic image subjected to the process of step S11.

As described above, according to the present example embodiment, for example, when the examination by the scope-type endoscope is performed, the information relating to the lesion candidate can be displayed without performing the examination by the capsule-type endoscope or the like in advance. Further, according to the present example embodiment, for example, during the endoscopic examination, the information relating to the lesion candidates can be displayed without performing processing such as acquiring information of the position of the lesion candidates while estimating the three-dimensional shape of the colon. Further, according to the present example embodiment, it is possible to display, as the information relating to the lesion, the information relating to the position of the lesion candidate in the endoscopic image, the information relating to the position in the colon at which the lesion candidate is detected, and the information relating to the risk of the lesion candidate. According to this display, the operator can reliably detect malignant lesions that are likely to occur on the right side of the colon. Therefore, according to this example embodiment, at the time of the endoscopic examination, it is possible to conveniently display information relating to the lesion candidates.

### Second Example Embodiment

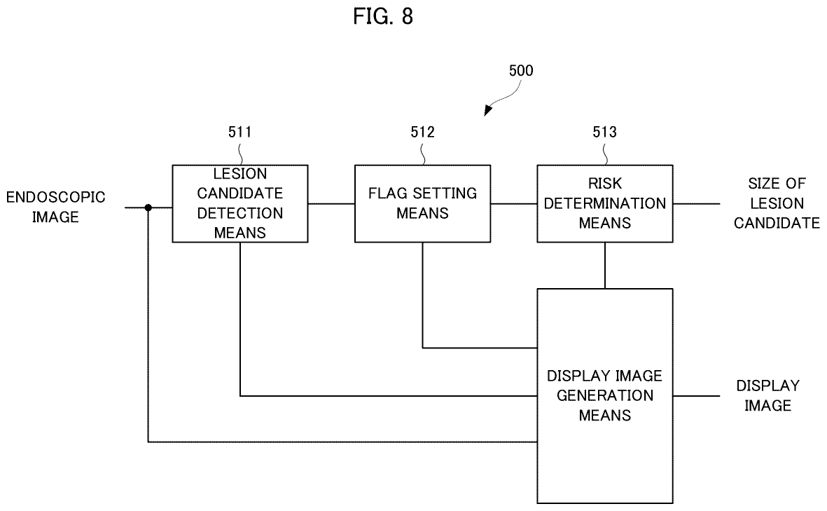

FIG. 8 is a block diagram illustrating a functional configuration of an endoscopic examination support apparatus according to a second example embodiment.

The endoscopic examination support apparatus 500 according to this example embodiment has the same hardware configuration as the endoscopic examination support apparatus 10. Further, the endoscopic examination support device 500 includes a lesion candidate detection means 511, a flag setting means 512, a risk determination means 513, and a display image generation means 514.

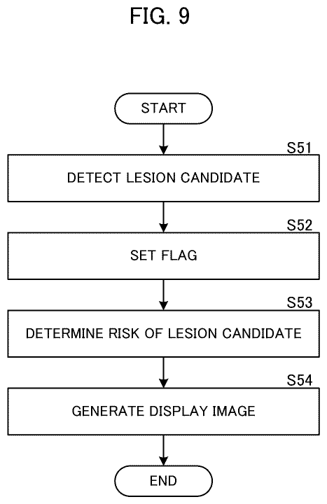

FIG. 9 is a flowchart for explaining processing performed in the endoscopic examination support apparatus according to the second example embodiment.

The lesion candidate detection means 511 detects a lesion candidate from an endoscopic image acquired by imaging an interior of a luminal organ (step S51).

The flag setting means 512 sets a flag according to a position in the interior of the luminal organ at which the endoscopic image including the lesion candidate is acquired (step S52).

The risk determination means 513 determines a risk of the lesion candidate based on at least a size of the lesion candidate and the flag (step S53).

The display image generation means 514 generates a display image including information relating to a position of the lesion candidate in the endoscopic image, information relating to a position in the interior of the luminal organ corresponding to the flag, and information relating to the risk of the lesion candidate (step S54).

According to this example embodiment, at the time of the endoscopic examination, it is possible to conveniently display information relating to the lesion candidates.

A part or all of the example embodiments described above may also be described as the following supplementary notes, but not limited thereto.

### (Supplementary Note 1)

An endoscopic examination support apparatus comprising:
a lesion candidate detection means configured to detect a lesion candidate from an endoscopic image acquired by imaging an interior of a luminal organ;
a flag setting means configured to set a flag according to a position in the interior of the luminal organ at which the endoscopic image including the lesion candidate is acquired;
a risk determination means configured to determine a risk of the lesion candidate based on at least a size of the lesion candidate and the flag; and
a display image generation means configured to generate a display image including information relating to a position of the lesion candidate in the endoscopic image, information relating to a position in the interior of the luminal organ corresponding to the flag, and information relating to the risk of the lesion candidate.

### (Supplementary Note 2)

The endoscopic examination support apparatus according to Supplementary note 1,
wherein the flag setting means sets a first flag in a period from a time when pull-out of the endoscope camera inserted into the interior of the luminal organ in order to acquire the endoscopic image is started to a time when the endoscope camera passes through a predetermined site in the luminal organ, and sets a second flag in a period from a time when the endoscope camera passes through the predetermined site to a time when pull-out of the endoscope camera is completed, and
wherein the risk determination unit determines that the risk of the first lesion candidate is equal to or larger than the risk of the second lesion candidate when the size of the first lesion candidate included in the endoscopic image corresponding to the first flag and the size of the second lesion candidate included in the endoscopic image corresponding to the second flag are equal.

### (Supplementary Note 3)

The endoscopic examination support apparatus according to Supplementary note 2, wherein the risk determination unit determines that the risk of the first lesion candidate is equal to or larger than the risk of the second lesion candidate when the size of the first lesion candidate and the size of the second lesion candidate are equal and the type of the first lesion candidate and the type of the second lesion candidate are same.

### (Supplementary Note 4)

The endoscopic examination support device according to Supplementary note 2 or 3, wherein, in a case where the luminal organ is colon, the flag setting means sets the first flag in a period from a time when the endoscope camera reaches a cecum to a time when the endoscope camera passes a splenic curvature, and sets the second flag in a period from a time when the endoscope camera passes through the splenic curvature to a time when the endoscope camera passes through an anus.

### (Supplementary Note 5)

The endoscopic examination support apparatus according to Supplementary note 1, wherein the display image generation means generates, as the display image, an image including a detection frame which surrounds the lesion candidate present in the endoscopic image and is displayed in different manners according to the flag and the risk of the lesion candidate.

### (Supplementary Note 6)

The endoscopic examination support apparatus according to Supplementary note 5, wherein the display image generation means generates, as the detection frame, a frame having different line types according to the flag.

### (Supplementary Note 7)

The endoscopic examination support apparatus according to Supplementary note 5, wherein the display image generation means generates, as the detection frame, a frame having different colors according to the risk of the lesion candidate.

### (Supplementary Note 8)

The endoscopic examination support apparatus according to Supplementary note 1, wherein the display image generation means generates, as the display image, an image including a detection frame surrounding the lesion candidate present in the endoscopic image, and a tag added to the detection frame to display different information according to the flag and the risk of the lesion candidate.

### (Supplementary Note 9)

An endoscopic examination support method comprising:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate.

### (Supplementary Note 10)

A recording medium storing a program, the program causing a computer to execute:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate.

While the present disclosure has been described with reference to the example embodiments, the present disclosure is not limited to the above example embodiments and examples. Various changes which can be understood by those skilled in the art within the scope of the present disclosure can be made in the configuration and details of the present disclosure.

### DESCRIPTION OF SYMBOLS

10 Endoscopic examination support apparatus
21 Candidate area detection unit
22 Flag setting unit
23 Lesion size classification unit
24 Lesion type classification unit
25 Lesion risk determination unit
26 Display image generation unit

What is claimed is:

1. An endoscopic examination support apparatus comprising:
a memory configured to store instructions; and
a processor configured to execute the instructions to:
detect a lesion candidate from an endoscopic image acquired by imaging an interior of a luminal organ;
set a flag according to a position in the interior of the luminal organ at which the endoscopic image including the lesion candidate is acquired;
determine a risk of the lesion candidate based on at least a size of the lesion candidate and the flag; and
generate a display image including information relating to a position of the lesion candidate in the endoscopic image, information relating to a position in the interior of the luminal organ corresponding to the flag, and information relating to the risk of the lesion candidate,
wherein the processor sets a first flag in a period from a time when pull-out of the endoscope camera inserted into the interior of the luminal organ in order to acquire the endoscopic image is started to a time when the endoscope camera passes through a predetermined site in the luminal organ, and sets a second flag in a period from a time when the endoscope camera passes through the predetermined site to a time when pull-out of the endoscope camera is completed, and
wherein the processor determines that the risk of a first lesion candidate is equal to or larger than the risk of a second lesion candidate when the size of the first lesion candidate included in the endoscopic image corresponding to the first flag and the size of the second lesion candidate included in the endoscopic image corresponding to the second flag are equal.

2. (canceled)

3. The endoscopic examination support apparatus according to claim 1, wherein the processor determines that the risk of the first lesion candidate is equal to or larger than the risk of the second lesion candidate when the size of the first lesion candidate and the size of the second lesion candidate are equal and the type of the first lesion candidate and the type of the second lesion candidate are same.

4. The endoscopic examination support device according to claim 1, wherein, in a case where the luminal organ is colon, the processor sets the first flag in a period from a time when the endoscope camera reaches a cecum to a time when the endoscope camera passes a splenic curvature, and sets the second flag in a period from a time when the endoscope camera passes through the splenic curvature to a time when the endoscope camera passes through an anus.

5. The endoscopic examination support apparatus according to claim 1, wherein the processor generates, as the display image, an image including a detection frame which surrounds the lesion candidate present in the endoscopic image and is displayed in different manners according to the flag and the risk of the lesion candidate.

6. The endoscopic examination support apparatus according to claim 5, wherein the processor generates, as the detection frame, a frame having different line types according to the flag.

7. The endoscopic examination support apparatus according to claim 5, wherein the processor generates, as the detection frame, a frame having different colors according to the risk of the lesion candidate.

8. The endoscopic examination support apparatus according to claim 1, wherein the processor generates, as the display image, an image including a detection frame surrounding the lesion candidate present in the endoscopic image, and a tag added to the detection frame to display different information according to the flag and the risk of the lesion candidate.

9. An endoscopic examination support method comprising:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate,
wherein a first flag is set in a period from a time when pull-out of the endoscope camera inserted into the interior of the luminal organ in order to acquire the endoscopic image is started to a time when the endoscope camera passes through a predetermined site in the luminal organ, and a second flag is set in a period from a time when the endoscope camera passes through the predetermined site to a time when pull-out of the endoscope camera is completed, and
wherein the risk of a first lesion candidate is determined to be equal to or larger than the risk of a second lesion candidate when the size of the first lesion candidate included in the endoscopic image corresponding to the first flag and the size of the second lesion candidate included in the endoscopic image corresponding to the second flag are equal.

10. A non-transitory computer-readable recording medium storing a program, the program causing a computer to execute:
detecting a lesion candidate from an endoscopic image acquired by imaging an interior of a luminal organ;
setting a flag according to a position in the interior of the luminal organ at which the endoscopic image including the lesion candidate is acquired;
determining a risk of the lesion candidate based on at least a size of the lesion candidate and the flag; and
generating a display image including information relating to a position of the lesion candidate in the endoscopic image, information relating to a position in the interior of the luminal organ corresponding to the flag, and information relating to the risk of the lesion candidate,
wherein a first flag is set in a period from a time when pull-out of the endoscope camera inserted into the interior of the luminal organ in order to acquire the endoscopic image is started to a time when the endoscope camera passes through a predetermined site in the luminal organ, and a second flag is set in a period from a time when the endoscope camera passes through the predetermined site to a time when pull-out of the endoscope camera is completed, and
wherein the risk of a first lesion candidate is determined to be equal to or larger than the risk of a second lesion candidate when the size of the first lesion candidate included in the endoscopic image corresponding to the first flag and the size of the second lesion candidate included in the endoscopic image corresponding to the second flag are equal.

11. The endoscopic examination support apparatus according to claim 1, wherein the processor sets the flag using a machine learning model trained to output data used to identify the position in the colon corresponding to the image including a subject in the colon in response to the input of the image.

12. The endoscopic examination support apparatus according to claim 1, wherein the display image is displayed as a guide to the risk of the lesion candidate to support decision making of a doctor.
